(19) United States
(12) Patent Application Publication
(10) Pub. No.: US 2007/0135706 A1
(43) Pub. Date: Jun. 14, 2007

(54) Debridement method, device and kit

(76) Inventors: Daniel A. Shimko, Collierville, TN (US); Stanley W. Olson JR., Germantown, TN (US); Jeffrey H. Nycz, Collierville, TN (US)

Correspondence Address: PHILIP D FREEDMAN PC;PHILIP D FREEDMAN, 317 S. FAYETTE STREET, ALEXANDRIA, VA, 22314-5902, US

(21) Appl. No.: 11/299,673

(22) Filed: Dec. 13, 2005

(51) Int. Cl. A61B 5/05

(52) U.S. Cl. 600/411

(57) ABSTRACT

A debridement method, device and system or kit are effectively used to debride a lesion, particularly an osteolytic lesion resulting from a hip or knee arthroplasty. In the lesion treatment, an effective amount of a debridement fluid with suspended particulate abrasive is delivered to a lesion area within body tissue to debride the lesion; and the fluid is intermittently aspirated from the area.

### BACKGROUND OF THE INVENTION

The invention relates to a method, device and kit for washing and debridement of a lesion. More particularly, the invention relates to an irrigation method, device and kit for debridement of an osteolytic lesion.

Osteolysis is a common complication in total hip arthroplasty and a common cause of component failure. Osteolysis is a response to wear debris. It can develop around a hip or knee implant as a result of the presence of bearing surface wear debris, access of wear debris particles to an implant-bone interface and a biologic osteolytic response of a host bone to debris laden synovial or other physiological fluids to the wear particles. Osteolysis is mediated primarily by macrophages. Fibroblasts and endothelial cells also play a role. These cells are activated by the bearing surface wear debris, primarily polyethylene, but also metal and polymethylmethacrylate particles. The biologic reaction to these particles is a nonspecific foreign-body reaction. Particles in the submicron size range undergo phagocytosis by macrophages and release a variety of cytokines which ultimately stimulate osteoclasts to resorb bone. The most common source of wear debris is adhesive-abrasive wear between a femoral head and polyethylene liner. This wear can produce as many as 500,000 particles per gait cycle.

Osteolysis can be asymptomatic until the lesions become very large. While some osteolytic lesions may be cleansed by washing and conventional debridement, surgery is a typical treatment. The surgery both treats the lesions and removes particles with attendant biofilm that could generate recurrence. With a stable acetabular component in acceptable alignment and with a modular liner, debridement and bone grafting of the lesions with retention of the acetabular shell and replacement of the polyethylene liner can be successful. However, if the acetabular shell is loose or malpositioned, then revision of the component is indicated.

While washing and debridement procedures are preferred approaches to lesion management, these less invasive procedures are not uniformly successful. Lesions can be difficult to debride, particularly osteolytic lesions. Osteolytic lesions are often located in tortuous and remote anatomy that is difficult to access using traditional instruments and these lesions are often filled with obstructing bony spicules, gelatinous masses of necrotic and fibrous tissue. This tissue can be adherent or non-adherent to surrounding intact tissue that defines the border of the lesion.

There is a need for an improved debridement method for osteolytic bone lesions that is minimally invasive and that does not require removal of a well-fixed previous implant. There is a need for a debridement method to effectively debride lesions in difficult anatomic locations and a need for a method to effectively break up soft tissue, clean the lesion edge and evacuate lesions that result with hip or knee implant procedures. There is a need for a device capable of breaking up the soft tissue, cleaning the lesion edge, removing the biofilms and evacuating the area through a substantially non-evasive arthroscopic methodology.

### BRIEF DESCRIPTION OF THE INVENTION

The invention relates to a debridement method, device and system or kit to effectively debride a lesion, particularly an osteolytic lesion resulting from a hip or knee arthroplasty. According to the invention, a method for treatment of a lesion, comprises: delivering an effective amount of a debridement fluid with suspended particulate abrasive to a lesion area within body tissue to debride the lesion; and intermittently aspirating the fluid from the area.

In an embodiment, the invention is a method for removing unwanted material from a body cavity comprising: providing a fluid reservoir with abrasive particle-containing debridement fluid and a device comprising a tubular flexible line having a pickup end and an delivery/aspirator end, an inner cannula and an outer second cannula that extend concentric with one another longitudinally as part of the tubular flexible line; the inner cannula having at least one orifice at the delivery/aspirator end of the tubular flexible line to deliver the debridement fluid with suspended particulate abrasive from the fluid reservoir to a lesion area in need of debridement; and the outer cannula substantially open at the delivery/aspirator end of the tubular flexible line to aspirate fluid from the area; and delivering an effective amount of a debridement fluid with suspended particulate abrasive from the reservoir by the inner cannula into the lesion area to debride the lesion; and aspirating fluid from the area by the outer cannula.

Another embodiment comprises a device for treatment of an osteolytic lesion, comprising: a fluid reservoir; abrasive particle-containing debridement fluid contained within the fluid reservoir; and a tubular conduit having a pickup end and delivery/aspirator end; an inner cannula and an outer cannula extending concentric to one another longitudinally as part of the tubular conduit, the inner cannula having at least one orifice at the delivery/aspirator end of the tubular conduit to deliver or aspirate debridement fluid with suspended particulate abrasive to or from a lesion area in need of debridement; and the outer cannula substantially open at the delivery/aspirator end of the tubular flexible line to deliver or aspirate fluid from the area.

In yet another embodiment, the invention is a kit or system for treatment of a lesion, comprising: a fluid reservoir; abrasive particle-containing debridement fluid contained within the fluid reservoir; a tubular conduit having a pickup end and delivery/aspirator end an inner cannula and an outer cannula, extending concentric to one another longitudinally as part of the tubular conduit; the inner cannula having at least one orifice at the delivery/aspirator end of the tubular conduit to deliver debridement fluid with suspended particulate abrasive from the fluid reservoir to a lesion area in need of debridement; and the outer cannula substantially open at the delivery/aspirator end of the tubular flexible line to aspirate fluid from the area; and an imaging device to monitor delivery of the debridement fluid to the lesion area and aspiration of fluid from the area.

### BRIEF DESCRIPTION OF THE DRAWINGS

FIG. 1 is a schematic elevation of a lesion debridement device;

FIG. 2 is a cross-sectional side view of a tubular flexible delivery tube end of the FIG. 1 device;

FIG. 3 is a schematic side elevation of a pulse-generating mechanism for the debridement device;

FIG. 4 is a schematic perspective view of a user using a system or kit including a lesion debridement device and monitoring fluoroscope; and

FIG. 5 shows a hip joint in need of treatment for a lesion and placement of a debridement device to effect irrigation of the lesion

### DETAILED DESCRIPTION OF THE INVENTION

In the invention, a lesion is irrigated with a fluid with suspended particulate abrasive by an irrigation process, preferably by pulse irrigation (also called “pulse lavage”).

Pulse lavage or pulse irrigation is one procedure for wound and lesion management. In this procedure, pulsating water is directed toward the wound or lesion area. This procedure is effective in removing debris and bacteria from wound and lesion areas. Pulse irrigation is used as part of a number of orthopedic procedures such as prosthetic joint replacement, in which it is used to remove bone fragments from an area of prosthesis. A controllable pulsating stream of liquid to a wound or lesion can provide a therapeutic action that promotes healing and decreases infection.

Various fluids are used with lavage or irrigation procedures to induce proper healing. In McCarthy U.S. Pat. No. 3,288,140, for example a method and apparatus for treatment of surface wounds by fluids is disclosed. Saline is the McCarthy preferred fluid. Further, Vad U.S. Pat. No. 6,527,760 uses normal saline in combination with an antibiotic

The debridement fluid of the invention can be water and other aqueous compositions, including any other typical irrigating or debridement solution. Preferably the fluid is a clear biocompatible debridement fluid such as warm isotonic saline or normal saline in combination with an antibiotic. However, many variations are possible. The solution may include buffers and a bicarbonate, citric acid and tanic acid in very low concentrations. Or the fluid can be a gas and liquid mixture. The gas can be oxygen or carbon dioxide or hydrogen peroxide useful for sterilization purposes. The fluid can include steroid and anti-inflammatory medicaments.

A preferred debridement fluid comprises a mixture of inorganic salts and, optionally minerals, compounded to mimic an electrolyte concentration and a body fluid mixture in an isotonic state. The fluid typically comprises a halide salt of lithium, sodium, potassium, calcium, and other cations. Typically the halide is fluoride, chloride, bromide, or iodide, and most typically chloride. A typical electrolyzed solution of the present invention has a pH within the range of about 2 to about 5, an oxidation reduction potential within the range of about +600 mV to about +1200 mV, and hypohalous acid concentration in the range of about 10 ppm to about 200 ppm. The solution can have bactericidal, fungicidal, and sporicidal properties.

The particulate abrasive can be a biosorable material, which preferably dissolves within several days. Preferably, the abrasive is resorbable and capable of passing through small gauge needles under lavage pressure. Calcium sulfate (CaSO₄) is a preferred material. The particulate abrasive can be present in the debridement fluid in a percent by weight between 0.1% and 65%; desirably between 1% and 40% and preferably between 3% and 15%.

Other possible bioabsorbable materials can be injectable solid forms of: calcium phosphate, tri-calcium phosphate, hydroxyapatite, coral hydroxyapatite, demineralized bone matrix, and mineralized bone matrix. Further, the bioabsorbable material can be an injectable solid form of a biopolymer, for example, polylactic acid, polyglycolic acid, polygalactic acid, polycaprolactone, polyethylene oxide, polypropylene oxide, polysulfone, polyethylene, polypropylene, hyaluronic acid or bioglass.

Though preferably the material is bioabsorbable, it is also possible that the material be merely bioimplantable, e.g., hydroxyapatite or PMMA. Material selection is based on the application. Hence, other abrasives may include calcium carbonate, perlite (an expanded silica abrasive), a colloid-forming clay, quartz, pumice, feldspar, tripoli and calcium phosphate, dextranomor microbeads, silicates of aluminum, calcium, lithium magnesium, lithium magnesium sodium, magnesium aluminum, magnesium, sodium and zirconium, attapulgite, bentonite, fuller's earth, hectorite, kaolin, montmorillonite, pyrophyllite, and zeolite. Other suitable particulate abrasives include biocompatible (resorbable and non-resorbable) ceramic and polymer particles such as hydroxyapatite, tetra-tri-calcium phosphate, tri-calcium phosphate, calcium sulfate, calcium aluminate and polymethylmethacrylate.

In some embodiments, particle size of the abrasive may be important. For example, in some applications, a fine particle size that forms a viscous suspension with a particular lavash fluid may be desirable, in other instances, the fluid may be too viscous for effective delivery to a lesion site. In some applications, where heavy abrasive may be desirable, in other instances, the particle size may be too large to pass through the orifice of a delivery device. The abrasive useable in the invention is of a particulate size as to be capable of passing through small gauge needles such as arthroscopic size syringes like the injection syringe of a device of the invention. The particulate abrasive in water preferably is of an average particle size between 0.1 microns and 1500; desirably between 10 microns and 1000 microns and preferably between 50 microns and 400 microns.

In an embodiment, the debridement fluid includes a proteolytic enzyme (protease) or chemonucleolytic component to further disrupt the matrix of lesion tissue. Suitable enzymes include vibriolysin, krill protease, chymotrypsin, trypsin, collagenase, elastase, dipase, proteinase K, Clostridium multifunctional protease, chymopapain, trypsin, chondroitinase, collagenase, Bacillus subtillis protease or a chemical, such as ethylenediaminetetraacetic acid (EDTA). These proteases are typically employed in therapeutic methods, demonstrate low incidence of undesirable side effects and are commercially available in pure, purified or genetically engineered forms. Other suitable proteases include papain, bromelain, plasminogen activator, plasmin, mast cell protease, lysosomal hydrolase, streptokinase, pepsin, and any or all fungal, bacterial, plant or animal proteases. In this embodiment, the debridement fluid may contain a single protease or a plurality of proteases. These additives are helpful when addressing biofilm or tissue remnants that are in difficult to access areas or areas in which a biofilm or remnant tissue is tightly adhered to the osteolytic lesion or to orthopeadic implant

An embodiment of the invention comprises following progress of the lesion debridement by fluoroscopy. In this embodiment, contrast agent is injected into the lesion area through a catheter, or preferably through the inner expression cannula of the device of the invention along with debridement fluid. In an example, the debridement instrument is inserted directly into the lesion site. The contrast agent migrates so that the lesion can be radiographically imaged with a fluoroscope. The fluoroscope produces a planar (or two dimensional) image of the lesion area that can be evaluated to monitor the debridement method.

Features of the invention will become apparent from the drawings and following detailed discussion, which by way of example without limitation describe preferred embodiments of the invention.

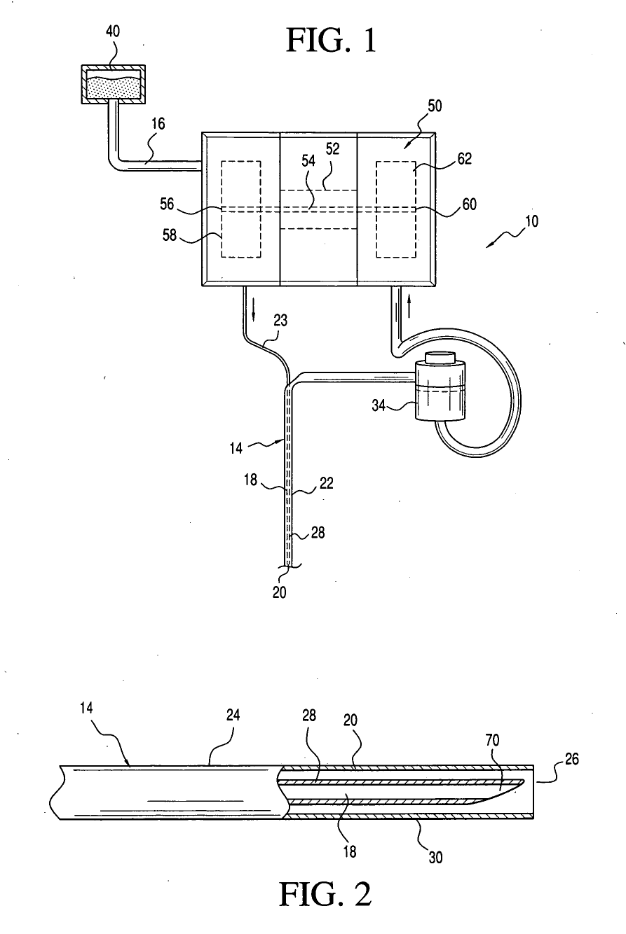

FIG. 1 shows an invention embodiment comprising a debridement device 10 for the washing and debridement of wounds and lesions of a patient. The system 10 includes housing 12 with conduit 14 for the delivery of fluid under pressure. With reference to FIGS. 1 and 2, inner expression cannula 18 and outer aspirator circumferential cannula 20 are shown longitudinally form the conduit 14. The conduit 14 includes a flexible pickup section 22 and a rigid delivery section 24. The system 10 includes a pressurized lavash fluid reservoir 40 and a fluid transfer pump 50, which is in fluid communication with inner expression cannula 18 and outer aspirator cannula line 20.

The conduit 14 has a pickup end 16 at lavash fluid reservoir 40 to operatively connect the inner cannula 18 from the reservoir 40 (through fluid transfer pump 50) to fluid aspirator/expression end 26 of rigid section 24. The outer aspirator cannula 20 is operatively connected from the fluid transfer pump 50 to fluid delivery/aspirator end 23 to fluid aspirator/discharge end 26 of rigid section 24. In this example, the fluid within the reservoir 40 is a saline solution. The saline solution comprises 10 weight percent suspended calcium sulfate particulate having a particle size of about 150 microns.

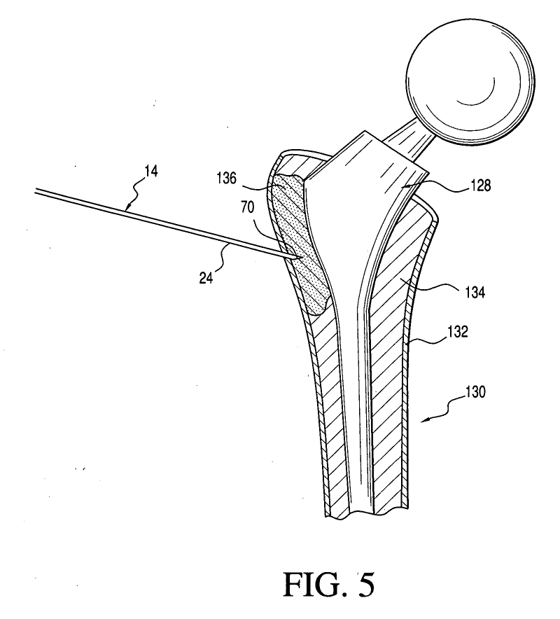

Fluid transfer pump 50 includes a drivable motor 52 having an elongated rotor shaft 54. A fluid pressure generating pump 58 is arranged at a first end 56 of the rotor shaft 54. The pump 58 provides fluid pressure to the dual cannula flexible tube 22 from reservoir 40. A second end 60 of rotatable shaft 54 is attached to a suction pump 62, also located within the housing 12. Suction pump is in fluid communication with a screened disposable collection bottle 34 to provide a vacuum incentive for drainage of fluids to the bag 34. In this embodiment, a common empowered motor 52 with an extended shaft 54 provides drive for both pressure pump 58 and vacuum source 62. The arrangement provides for a dual continuous pulsed feed of fluid to a patient lesion area shown in FIGS. 5 for a continuous withdrawal of fluid from the area after treatment of a wound or lesion.

FIG. 2 is a cut away depiction of rigid delivery section 24 of the conduit 14 including inner cannula 18 and outer cannula 20. While the section 24 is described as “rigid” it can be a flexible articulating section as well. The section 24 can be of any material that resists degradation from the expressing particles. Inner cannula 18 provides a passageway for lavash fluid from fluid reservoir 40. The fluid is expressed from syringe end 70 of the inner cannula 18 to a wound or lesion area. An outer wall 30 of conduit 14 forms outer cannula 20 with wall 26 of inner cannula 18 to provide a fluid passageway for aspirating fluid from wound or lesion area after lavage treatment. While this description identifies inner cannula 18 as a passageway to deliver the fluid and outer cannula 20 as a passageway to aspirate, the invention covers other configurations of the conduit 14. For example, outer cannula 20 can be configured to deliver fluid, while inner cannula 18 aspirates.

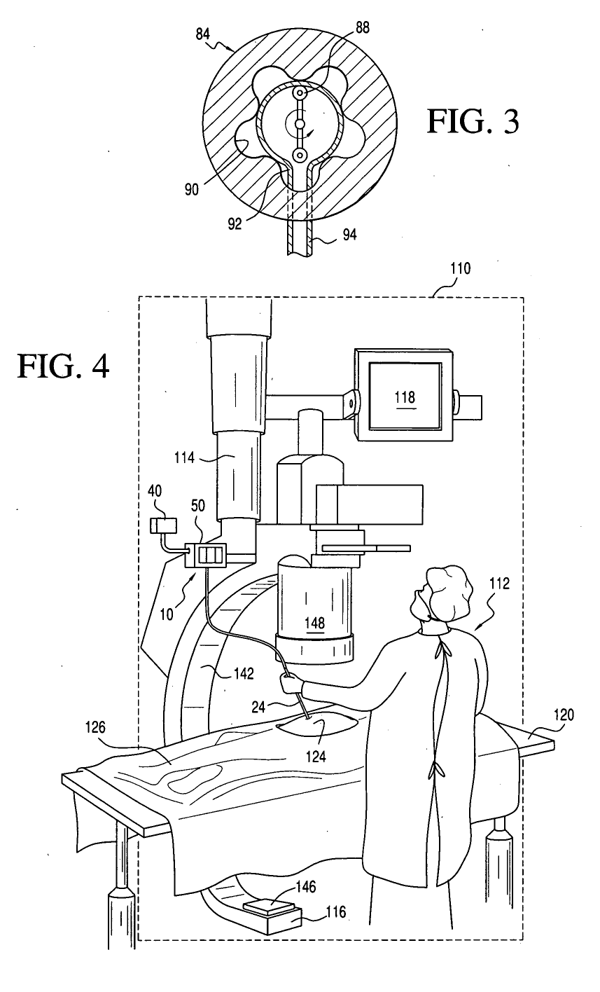

In an embodiment shown in FIG. 3, pulsating pump 84 has a rotating wheel 88 arranged to spin within sinusoidal inner surface 90. The sinusoidal operation of the wheel 88 intermittently squeezes and releases flexible fluid feedline 92. Feedline 92 includes pickup end 16 at fluid source 40 (shown in FIG. 1). A fluid feed section 96 extends to form inner expression cannula 18, shown in FIG. 1. Rotation of wheel 88 within the sinusoidal surface 90 generates intermittent pulses that are discharged through the pressured inner expression cannula 18 to be expressed at syringe end 70. In an embodiment, the suction side of the fluid transfer pump 50 is effected in a pulsed manner similar to the fluid pressure side. The suction or vacuum side 62 of the pump 50 can be in-phase or out-of-phase with the fluid pressure pulsating pump 58.

FIG. 5 shows a hip joint in need of treatment for a lesion 136 and placement of aspirator/expression end 26 of the debridement device 10 to effect irrigation of the lesion 136. Further, FIG. 4 illustrates fluoroscopic monitoring of the debridement.

First, referring to FIG. 4, a user 112 is shown using a system or kit (delineated by dashed outline 110) including a support member 114 supporting a monitoring fluoroscope 116, an image display 118 such as a flat panel television monitor and a lesion debridement device 10. The user 112 grasps the rigid delivery section 24 of the debridement device 10 and inserts it into a hip joint 124, shown interiorly in FIG. 5, of a patient (the patient's outline beneath a sheet is indicated at 126). FIG. 5 shows a hip implant 128 that has been surgically implanted into the proximal femur (hip) 130. The implant 128 may be of any form; for example, fixed, modular, primary, revision, ceramic head or metal head. In non-diseased portions of hip 130, implant 128 is well-fixed between cortical bone 132 and cancellous bone 134. In a diseased portion of hip 130, osteolytic lesion 136 takes up space that would normally be filled with cancellous bone 134. Lesion 136 is often soft and spongy. Though lesion 136 is depicted in this embodiment as being in the area of the proximal stem, it could be in the area of the distal stem or in another area.

Usually lesion 136 is surrounded by cancellous bone 134, and usually also cortical bone 132. And, typical treatment to debride the lesion 136 is significant and invasive, sometimes involving removal of the implant 128, open debridement of the lesion area 136 (which enlarges the intramedullary area even further), and implantation of a revision implant. In another typical treatment, location of the lesion 136 is identified by fluoroscope or other imaging process, first and second holes are bored to access the lesion area and lavage fluid is expressed through one hole and is suctioned out the second hold. This procedure operates blindly without assurance that fluid expressed through the first hole delivers lavage to the lesion area. Additionally, the lesion can be tough and resistant to a typical fluid that would be used in the first and second hole procedure.

The present invention provides a minimally-invasive and accurate approach to treating lesions without removal of implants and revision and without two hole bodily invasion. The invention accurately delivers lavage to assure complete debridement of the lesion. In the present invention, a lavage fluid is utilized that comprises abrasive particles that completely debride even an osteolytic lesion that may be filled with resistant gelatinous masses of nacrotic and fibrous tissue. Additionally, in an embodiment of the invention, insertion of the rigid delivery section 24 of the debridement device into the hip joint, the orientation of the syringe expressing end 70 of the delivery section 24; impingement of expressed debridement fluid the lesion and aspirating of fluid containing the nacrotic and fibrous tissue and spent fluid and particles can be monitored to assure complete debridement.

The lesion debridement is monitored in FIG. 4 by viewing a fluoroscopic image of the hip joint 124, lesion area 136, and inserted rigid delivery section 14. The patient 126 resides on table 120, which is essentially transparent to x-rays. A support member 122 supports a fluoroscope and a television monitor 118. The fluoroscope 116 can be supported by a C-shaped arm 142 device, as shown. Table 120 and patient 126 are positioned within the C formed by arm 142. Fluoroscope 116 is an x-ray tube unit at a lower end of the C-shaped arm. The x-ray tube unit 116 emits an x-ray beam in a generally upward vertical direction through a diaphragm 146. The x-ray beam is directed upward through the table 120 and the hip joint 124 of patient 126. The x-ray beam is received by image intensifier 148, which includes a television camera (not shown). A fluoroscopic field of view received by the camera at image intensifier 148 is projected on television monitor 118.

In operation, patient 126 is aligned between tube unit 116 and image intensifier 148 so that the internal patient's hip joint 124 is visible on television monitor 116. User 112 performs a puncture of the patient's hip area toward the joint 124 with the elongated rigid delivery section 24 of debridement device 10. The user 112 positions the puncture so that the inserted delivery section 24 syringe end is generally perpendicular to a central axis of an x-ray beam, which is directed upward from fluoroscope x-ray tube unit 116 to image intensifier 148. The fluoroscopic field of view of fluoroscope 116 is then narrowed to display an image on monitor 116 to permit positioning aspirator/expression end 26 of delivery section 24 within the cancellous bone 134 of hip joint 124 at a location of the osteolytic lesion 136.

The user 112 manipulates the aspirator/expression end 26 of delivery section 24, while remaining outside of the path of the x-ray beam between x-ray tube unit 116 and image intensifier 148 as shown in FIG. 4. The user 112 views the location and orientation of aspirator/expression end 26 of delivery section 24 on television monitor 116 while activating the pulse lavage action of the debridement device 20. Throughout the procedure, the user 112 monitors the location and orientation of the aspirator/expression end 26 to express the particulate abrasive-containing lavage fluid from reservoir 40. In an embodiment, the user 112 delivers the debridement fluid and aspirates the fluid by alternating pulse lavage. This procedure effectively debrides the lesion 136 and intermittently aspirates resistant osteolytic lesion constituents including nacrotic and fibrous tissue and spent particulate abrasive-containing lavage fluid.

While preferred embodiments of the invention have been described, the present invention is capable of variation and modification and therefore should not be limited to the precise details of the above examples. For example, the cannulas of the drawings are shown concentric. However, they can be side by side or of any suitable configuration. Also, the invention can relate to a kit that is packaged to include the above-described components for sale, shipment. The invention includes changes and alterations that fall within the purview of the following claims.

What is claimed is:

1. A method for treatment of a lesion, comprising:
delivering an effective amount of a debridement fluid with suspended particulate abrasive to a lesion area within body tissue to debride the lesion; and
intermittently aspirating the fluid from the area.

2. The method of claim 1, comprising delivering the debridement fluid and intermittently asperating the fluid by pulse lavage.

3. The method of claim 1, further comprising: injecting a radio-opaque contrast agent to the lesion area along with the debridement fluid.

4. The method of claim 1, comprising imaging the delivering of the fluid with suspended particulate abrasive and adjusting the delivering according to the imaging to direct the fluid with suspended particulate to a lesion area in need of debridement.

5. The method of claim 1, further comprising injecting a proteolytic enzyme to the lesion area along with the debridement fluid.

6. A device for treatment of an osteolytic lesion, comprising:
a fluid reservoir;
abrasive particle-containing debridement fluid contained within the fluid reservoir; and
a tubular conduit having a pickup end and delivery/aspirator end and first and second cannulas extending with one another longitudinally as part of the tubular conduit; the first cannula having at least one orifice at the delivery/aspirator end of the tubular conduit to deliver or aspirate debridement fluid to or from the distinguished lesion; and the second cannula substantially open at the delivery/aspirator end of the tubular conduit to deliver or aspirate fluid to or from the distinguished lesion.

7. The device of claim 6, wherein the particulate containing debridement fluid comprises between 0.1 and 65 percent by weight particulate abrasive in water.

8. The device of claim 6, wherein the particulate containing debridement fluid comprises an average particle size particulate between 0.1 microns and 1500 microns and water.

9. The device of claim 6, wherein the particulate containing debridement fluid further comprises (i) a contrast medium, (ii) a thrombolytic medication administered to facilitate treatment of the lesion, (iii) a steroid medicament, (iv) an anti-inflammatory medicament, (v) a contrast agent, or (vi) a proteolytic enzyme.

10. The device of claim 6, wherein the particulate containing debridement fluid further comprises a mixture of inorganic salts compounded to mimic an electrolyte concentration and mixture of body fluid in an isotonic state.

11. The device of claim 6, wherein the particulate abrasive comprises a biosorable material.

12. The device of claim 6, wherein the particulate abrasive comprises calcium sulfate (CaSO₄).

13. A method for removing material from a lesion area comprising:
providing a fluid reservoir with abrasive particle-containing debridement fluid and a device comprising a tubular conduit having a pickup end and an delivery/aspirator end, an inner cannula and an outer second cannula that extend concentric with one another longitudinally as part of the tubular conduit; the inner cannula having at least one orifice at the delivery/aspirator end of the tubular conduit to deliver the debridement fluid with suspended particulate abrasive from the fluid reservoir to a lesion area in need of debridement; and the outer cannula substantially open at the delivery/aspirator end of the tubular flexible line to aspirate fluid from the area; and
delivering an effective amount of a debridement fluid with suspended particulate abrasive from the reservoir by the inner cannula into the lesion area to debride the lesion; and
aspirating fluid from the area by the outer cannula.

14. The method of claim 13, comprising:
directing the delivery/aspirator end of the tubular conduit into the vicinity of a body lesion area;
providing pressurized fluid to jet at least one fluid with suspended particulate abrasive from the tubular flexible line via the inner cannula to the lesion area to loosen unwanted material; and
aspirating unwanted material from the body lesion area into the outer cannula of the tubular flexible line for discharge to a disposable collection bottle.

15. The method of claim 13, further comprising:
injecting a contrast medium into the vicinity of the body lesion area to image a location of the lesion; and
delivering the fluid with suspended particulate abrasive and adjusting the position of the tubular flexible line according the location of the lesion area.

16. The method of claim 13, comprising imaging a position of a delivery cannula of the tubular conduit delivering the fluid with suspended particulate abrasive and adjusting the position of the delivery cannula according to the imaging to direct the fluid with suspended particulate abrasive into a lesion area in need of debridement, wherein the position of the delivery cannula with respect to the lesion area in need of debridement is imaged with an affixed radio-opaque marker under fluoroscopy.

17. The method of claim 13, comprising generating a real-time fluoroscopic image to monitor (i) insertion of the delivery/aspirator line into a hip joint, (ii) orientation of a syringe expressing end of the delivery section or (iii) impingement of expressed debridement fluid to the lesion and aspirating of fluid containing nacrotic and fibrous tissue and spent fluid and particles from the lesion.

18. A kit for treatment of a lesion, comprising:
a fluid reservoir;
abrasive particle-containing debridement fluid contained within the fluid reservoir;
a tubular conduit having a pickup end and delivery/aspirator end an inner cannula and an outer cannula, extending concentric to one another longitudinally as part of the tubular conduit; the inner cannula having at least one orifice at the delivery/aspirator end of the tubular conduit to deliver debridement fluid with suspended particulate abrasive from the fluid reservoir to a lesion area in need of debridement; and the outer cannula substantially open at the delivery/aspirator end of the tubular conduit to aspirate fluid from the area; and
an imaging device to monitor delivery of the debridement fluid to the lesion area and aspiration of fluid from the area.

19. The kit of claim 18, wherein the imaging device comprises a fluoroscope that includes an x-ray source oriented to emit x-rays toward the lesion area; a radiation detector that detects x-rays from the source that have traversed the lesion area; an image display to generate a real-time fluoroscopic image showing the relationship of the delivery/aspirator end of the tubular conduit to the area on a display monitor from signals that are responsive to the detected x-rays.

20. The kit of claim 18, wherein the abrasive particle-containing debridement fluid contains a proteolytic enzyme.
